(19) United States
(12) Patent Application Publication
(10) Pub. No.: US 2003/0195186 A1
(43) Pub. Date: Oct. 16, 2003

(54) Methods of treating medication-, substance-, disease-, and other medical condition-related sexual dysfunction

(75) Inventor: Nathan Andrew Shapira, Gainesville, FL (US)

Correspondence Address: Frank C. Eisenschenk, Ph.D., Saliwanchik, LIoyd & Saliwanchik, Suite A-1, 2421 N.W. 41Street, Gainesville, FL, 32606, US

(73) Assignee: University of Florida, Gainesville (US)

(21) Appl. No.: 10/411,644

(22) Filed: Apr. 10, 2003

Related U.S. Application Data
(60) Provisional application No. 60/371,666, filed on Apr. 10, 2002.

(51) Int. Cl. A61K 35/78; A61K 31/55; A61K 31/551; A61K 31/4745; A61K 31/47; A61K 31/137; A61K 31/407; A61K 31/445

(52) U.S. Cl. 514/214.03; 514/221; 514/252.16; 514/253.03; 514/297; 514/295; 514/411; 424/752; 514/283; 514/317; 514/220; 514/649

(57) ABSTRACT

Many males and females experience sexual dysfunction either caused or made worse by medications, other substances, diseases, and other medical conditions. Currently, there is need for additional treatment alternatives for these patients' sexual dysfunction. The subject invention provides a novel treatment for these individuals with sexual dysfunction by inhibiting the enzyme that breaks down acetylcholine (a compound that helps modulate normal sexual function) and elevates acetylcholine levels in the body.

### CROSS-REFERENCE TO RELATED APPLICATION

This application claims the benefit of provisional patent application Serial No. 60/371,666, filed Apr. 10, 2002, which is hereby incorporated by reference in its entirety, including all figures, tables, and drawings.

### BACKGROUND OF THE INVENTION

Numerous prescription or non-prescription medications, illicit drugs, substances (alcohol, cigarettes, environmental toxins, etc.), diseases (such as, but not limited to, diabetes, cardiovascular disease), and other medical conditions (such as prostate surgery) have been associated with causing, or contributing, to sexual dysfunction (1,2). There are an estimated 1.5 billion prescriptions written every year in the United States (3). This equates to about 6 prescriptions for every person (4). Furthermore, sexual dysfunction related to medications and other substances can lead to medication noncompliance (5,6).

Medication and substance-related sexual dysfunction encompasses a broad range of changes in sexual function including reduced sexual desire, delayed or inhibited ejaculation, delayed orgasm, anorgasmia, or erectile disorder (6). Examples of types of medications associated with higher rates of sexual dysfunction include anti-hypertensives, psychiatric prescription medications, nonprescription medications, illicit drugs, and other substances (1).

It is extremely common for patients to experience problems or concerns about sexual function. For example, approximately 15 to 25% of patients seen in family practice settings are estimated to have problems or concerns about sexual function (1,7). There are other estimates that, in the United States, between 20 and 30 million men are affected by erectile difficulties and the incidence of disease increases sharply with age (6).

Many hormones, neurotransmitters and neuropeptides in the periphery and the central nervous system are involved in the neurobiology of sexual function (3). One of these compounds, acetylcholine, has been implicated in modulation of sexual function (3). For example, the human corpus cavernosum (where smooth muscles must relax for blood to flow in for an erection) is innervated by cholinergic nerves (8,9) and contains cholinergic receptors (10,11). When acetylcholine chloride was exogenously added to pre-contracted corpus cavernosum tissue, the muscles relaxed (12). In terms of acetylcholine receptors there are two basic types: nicotinic and muscarinic based on the ability of natural alkaloids (muscarine and nicotine) to mimic the effects of acetylcholine. Each of these receptors responds to acetylcholine, but each are also responsive to different agonists and antagonists. The receptors are also located in different regions in the nervous system. The specific cholinergic receptors modulating relaxation of the corpus cavernosum tissue are thought to be muscarinic because these effects are blocked by atropine (13,14).

Diseases and other medical conditions that have been implicated in sexual dysfunction, and in particular male erectile disorders, are common and include diabetes, cardiovascular disorders, endocrine disorders, neurologic disorders, and status post surgical procedures; and these tend to worsen with age (2, 15-17). It is estimated that in the United States 2 million men are impotent due to diabetes mellitus, 1.5 million are impotent as a result of vascular disease, 300,000 due to endocrine diseases (other than diabetes), 180,000 due to multiple sclerosis, 400,000 due to traumas and fractures, and 650,000 due to radical surgeries including prostatectomies, colostomies, and cystectomies (2).

In diseases and other medical conditions that have been implicated in sexual dysfunction, acetylcholine may play a role. For example, acetylcholine-induced relaxation of the corpus cavernosum appears impaired in diabetes mellitus (18,19). Other diseases that have increased sexual dysfunction (9), such as atherosclerosis associated to hypercholesterolemia, have also been associated with impaired acetylcholine-induced relaxation in the corpus cavernosum (20,21).

Although cholinergic mechanisms are not felt to be the dominant mechanisms in sexual function, they may have important modulatory roles for other neurotransmission systems (6,22,23). For example, atropine (acetylcholine antagonist) does not block erectile response for males viewing sexually erotic material (23), and in females atropine and bethanechol do not appear to affect vaginal vasocongestion (24,25). Furthermore, injection of acetylcholine chloride into the blood supply of the penis does not produce an erection (26). However, in terms of the modulatory role for acetylcholine, there are, several case reports of the cholinergic agent, bethanechol (cholinergic agonist), being used to reverse antidepressant-induced erectile and ejaculation difficulties (27-29). In male rats, both cholinergic agonists and antagonists reduce sexual activity and increasing cholinergic activity leads to increased ejaculation rapidity (30,31). In human males, bethanechol has been reported to reverse protriptyline, imipramine, and phenelzine-induced anorgasmia (inability to ejaculate) (22,32,33). An adrenergic-cholinergic balance has also been hypothesized as important for erectile function (22). Erectile dysfunction has been reported restored with benztropine (anticholinergic) for a patient receiving the antipsychotic fluphenazine decanoate, which has adrenergic-blocking effects (34). Penile erection has also been shown to be mainly evoked by nitric oxide (35) and studies have implicated NO as having an important role in cholinergically-induced relaxation of cavernosal tissue in rabbits and humans (19,36).

Centrally, acetylcholine may also play a role in sexual function. For example, repetitive orgasms have been reported in a epileptic woman who received acetylcholine infused into her brain's septal region (37). Dopamine, particularly through D-5 receptors (D1-like receptor) has been shown to modulate acetylcholine release in male rat brains (38). Dopamine mechanisms have also been indicated to be involved in the up-regulation of muscarinic acetylcholine receptors by NMDA antagonists in mice (39). Dopamine agonists (apomorphine hydrochloride and levodopa) have been associated with some patients having increased sexual desire (40-43) and facilitation of erection (44-53). In animal studies with male rats, dopamine agonists have been shown to increase mounting behavior and increase sexual behavior in sexually satiated rats (54-57). Amphetamines that can increase sexual desire, particularly at low dosages, (1) are also associated with increased acetylcholine release in the cortex of rats (58). While more limited data exists for women, dopamine antagonists are associated with delayed or inhibited orgasm (59-61).

Many prescription and non-prescription medications, illicit drugs, and other substances have significant effects on the cholinergic system. For example, it has been suggested that the affinity of the antidepressant paroxetine for the cholinergic receptor (an anti-cholinergic substance) is a factor in the paroxetine associated erectile dysfunction (62). Additionally, anti-cholinergic effects of clomipramine may be associated with its associated sexual dysfunction, particularly erectile dysfunction (6). Other anti-cholinergics (such as benztropine, trihexyphenidyl, scopolamine, and atropine) have been associated with sexual dysfunction (1). Other examples of medications, illicit drugs, and substances associated with sexual dysfunction and negatively effect cholinergic function include amitriptylinoxide, doxepine, and clozapine in the rat caudatoputamen (63), cimetidine and ranitidine at some cholinergic sites (64), nicotinic acetylcholine receptors by fluoxetine in rat hippocampus (65) and sertraline, paroxetine, and venlafaxine on human and one chick nicotinic acetylcholine receptor subtype (66), long term administration of imipramine in rat cortex (67), in guinea-pig ileum (68), functional blockade of neuronal nicotinic acetylcholine receptors by buproprion in mice (69), fluphenazine in rat striatum (70), chronic intake of ethanol in humans and rats (71), opioids (particularly binding delta- and mu-types) in rat striatum and hippocampus (72), cigarette smoking and nicotinic acetylcholine receptor up-regulation in rats (73), barbiturates on nicotinic acetylcholine receptors in rat central nervous system (74), and lead exposure on central cholinergic function in rat (75).

Potent inhibitors of acetylcholinesterase are toxic for humans however there are acetylcholinesterase inhibitors which have been safely tested and used in humans (for Alzheimer's disease), and they can be classified as reversible (donepezil, galantamine, and tacrine), pseudo-reversible (eptastigmine, physostigmine, and rivastigmine) or irreversible (metrifonate) enzyme inhibitors (76). Donepezil hydrochloride, galantamine, rivastigmine, and tacrine are FDA approved in the United States for Alzheimer's Disease (77). An additional anticholinesterase inhibitor is neostigmine which is approved in the United States for symptomatic treatment of myasthenia gravis (77). There are also reports that Huperzine A (a Chinese herbal remedy for inflammation and fever) is an acetylcholinesterase inhibitor and effective in the treatment of Alzheimer's disease (78,79). Galantamine appears to also have a second mechanism related to acetylcholine whereby in addition to inhibition of acetylcholinesterase, it appears to interact allosterically with nicotinic acetylcholine receptors potentiating agonists at these receptors (80,81). Novel anticholinesterase inhibitors are also being developed (for example see 82-84). There are also reports of transdermal systems for delivery of acetylcholinesterase inhibitors that are being developed (for example see 85,86).

Several acetylcholinesterase inhibitors have been reported to have the side effect of increasing libido in some patients. For example, increased libido was reported in clinical trials for Alzheimer's Disease of donepezil (greater than 1/100 patients), galantamine (1/100 to 1/1000 patients), rivastigmine (1/100 to 1/1000 patients), and tacrine (1/100 to 1/1000 patients) (77). Injected physostigmine has been shown in rats to reverse inhibition of estrogen-activated lordosis by androgens (87) and there are case reports of subcutaneous physostigmine and intrathecal neostigmine assisting spinal-cord injured (paralyzed/paraplegic) male patients with anejaculation being able to obtain ejaculation in order to have children (88-90). Other than case reports for anejaculation in spinal cord injured males with physostigmine and neostigmine, there are no reports in the literature of acetylcholinesterase inhibitors being used to treat medication-, substance-, disease-, and other medical condition-related sexual dysfunction (reduced sexual desire, delayed or inhibited ejaculation, delayed orgasm, anorgasmia, or erectile disorder).

### BRIEF SUMMARY OF THE INVENTION

This subject invention provides for the administration of (optimally by oral [pill/tablet/powder or liquid], transdermal methods (including iontophoresis), or by nasal spray) a therapeutically effective amount of at least one acetylcholinesterase inhibitor for the treatment of medication-, substance-, disease-, and other medical condition-related sexual dysfunction. Non-limiting examples of such sexual dysfunction include, and are not limited to reduced sexual desire, delayed or inhibited ejaculation, delayed orgasm, anorgasmia, or erectile disorder in males and/or females. Acetylcholinesterase inhibitors in these populations can be taken either as needed (prn) before sexual activity (depending on the particular kinetics and absorption of the specific acetylcholinesterase inhibitor—for example, 30 minutes to 2 hours before sexual activity) or as a scheduled daily medication.

### BRIEF DESCRIPTION OF THE TABLES

Tables 1-3 provide questionnaires for the assessment of sexual function in males and females (drawn from Kennedy et al. (91)).

### DETAILED DESCRIPTION OF THE INVENTION

This subject invention provides for the administration of a therapeutically effective amount of at least one (or one or more) acetylcholinesterase inhibitor for the treatment of medication-, substance-, disease-, and/or other medical condition-related sexual dysfunction. Non-limiting examples of such sexual dysfunction include, and are not limited to, reduced sexual desire, delayed or inhibited ejaculation, delayed orgasm, anorgasmia, or erectile disorders. The subject invention also provides methods of inducing an intended pharmacological result in patients afflicted with medication-, substance-, disease-, and/or other medical condition-related sexual dysfunction comprising the administration of a therapeutically effective amount of one or more acetylcholinesterase inhibitor.

The invention is applicable to the treatment of sexual dysfunction in males and/or females. Acetylcholinesterase inhibitors in these populations can be taken either as needed (prn) before sexual activity (depending on the particular kinetics and absorption of the specific acetylcholinesterase inhibitor—for example, 30 minutes to 2 hours before sexual activity) or as a scheduled daily medication. Administration routes for the acetylcholinesterase inhibitors of the invention include, and are not limited to, oral (e.g., powder, pill, tablet, capsule, caplet, or liquid), transdermal (including, but not limited to, transdermal patches, ointments, salves, pastes, or iontophoresis), or nasal/muscosal routes (e.g., sprays or drops).

Diseases and other medical conditions suitable for treatment according to the subject invention include sexual dysfunction caused by cardiovascular disorders (e.g., atherosclerotic disease, hyperlipidemia, aortic aneurysm, Leriche's syndrome, and/or cardiac failure), neurologic disorders, status post surgical procedures, vascular disease, multiple sclerosis, physical trauma and fractures, radical surgeries (including, but not limited to prostatectomies, colostomies, and cystectomies), renal and urological disorders (such as Peyronie's disease, chronic renal failure, hydrocele and/or varicocele), hepatic disorders (such as cirrhosis or cirrhosis associated with alcohol dependence), pulmonary disorders (for example, asthma, allergy, or respiratory failure), genetics (e.g., Klinefelter's syndrome), menopause, nutritional disorders (e.g., malnutrition, vitamin deficiencies), endocrine disorders (e.g., diabetes mellitus, dysfunction of the pituitary-adrenal-testis axis, acromegaly, Addison's disease, chromophobe adenoma, adrenal neoplasia, myxedema, hyperthyroidism), cancer, neurological disorders (e.g., multiple sclerosis, Parkinson's disease, transverse myelitis, temporal lobe epilepsy, traumatic and neoplastic spinal cord disease, central nervous system tumor, amyotrophic lateral sclerosis, peripheral neuropathy, general paresis, tabes dorsalis), and surgical procedures (e.g., perineal prostatectomy, abdominal-perineal colon resection, sympathectomy, aortoiliac surgery, radical cystectomy, retroperitoneal lymphadenectomy, or hysterectomy).

In some aspects of the invention, the medication related to (or inducing) sexual dysfunction can be one or more, or a combination, of the following medications selected from the group consisting of: paroxetine, clomipramine, benztropine, trihexyphenidyl, scopolamine, atropine, amitriptylinoxide, doxepine, clozapine, caudatoputamen, cimetidine, ranitidine, fluoxetine, sertraline, paroxetine, venlafaxine, imipramine, buproprion, fluphenazine, opioids, barbiturates, amiloride, atenolol, benazepril, chlorthalidone, clonidine, diltiazem, enalapril, guanabenz, guanadrel, guanethidine, hydralazine, hydrochlorothiazide, indapamide, labetalol, lisinopril, mecamylamine, mehyldopa, metoprolol, metyrosine, minoxidil, phenoxybenzamine, phentolamine, pindolol, prazosin, propranolol, reserpine, spironolactone, timolol, trimethaphan, verapamil, amoxapine, buproprion, desipramine, citalopram, doxepin, fluoxetine, imipramine, maprotiline, nortriptyline, paroxetine, phenelzine, protriptyline, sertraline, tranylcypromine, trazodone, trimipramine, venlafaxine, alprazolam, barbiturates, buspirone, chlordiazepoxide, chlorpromazine, clomipramine, clonazepam, clozapine, diazepam, droperidol, fluphenazine, haloperidol, lithium, lorazepam, meprobamate, mesoridazine, molindone, oxazepam, perphenazine, pimozide, prochlorperazine, risperidone, sulpiride, thioridazine, thiothixene, trifluoperazine, acetazolamide, aminocaproic acid, amiodarone, atropine, baclofen, benztropine, biperiden, bromocriptine, carbamazepine, cimetidine, clidinium, clofibrate, cyclobenzaprine, danazol, dichlorphenamide, dicyclomide, digoxin, disopyramide, disulfiram, ethinyl estradiol, ethionamide, etretinate, famotidine, gemfibrozil, glycopyrrolate, heparin, homatropine methylbromide, hydrochlorothiazide, hydroxyzine, indomethacin, interferon, isotretinoin, ketoconazole, mazindol, meclizine, medroxyprogesterone, methadone, methazolamide, methotrexate, methysergide, metoclopramide, metronidazole, mexiletine, morphine, naproxen, niacin, nizatidine, norethindrone, omeprazole, orphenadrine, oxybutynin, phendimetrazine, phenobarbital, phentolamine, phenytoin, primidone, probucol, procarbazine, prochlorperazine, procyclidine, propantheline bromide, propofol, ranitidine, scopolamine, sulfasalazine, tamoxifen, testosterone, thiabendazole, trihexyphenidyl, and trimeprazine), illicit or abused drugs and other substances (including, but not limited to alcohol, high dose amphetamines, amyl nitrite, barbiturates, cocaine, diazepam, herbicides, lead, marijuana, MDMA, methaqualone, morphine, and tobacco) and various combinations thereof.

Exemplary drugs (e.g., acetylcholinesterase inhibitors) useful for the treatment of the aforementioned disorders can be selected from the group consisting of donepezil, galantamine, tacrine, eptastigmine, physostigmine, rivastigmine, metrifonate, neostigmine, huperzine A, and combinations thereof. In various embodiments, the drugs for treatment of the aforementioned disorders can be administered singly, in various combinations, or in various combinations of up to nine drugs. Acetylcholinesterase inhibitors can be used alone for medication-, substance-, disease-, and other medical condition-related sexual dysfunction, or acetylcholinesterase inhibitors in combination with other agents commonly being used for treating sexual dysfunction. These agents/medications include selective phosphodiesterase type-5 (PDE5) inhibitors, such as sildenafil, tadalafil, vardenafil; cyproheptadine; tradodone; nefazodone; mianserin; mirtazapine; granisetron; yohimbine; amantadine; dextroamphetamine; pemoline; methylphenidate; buproprion; buspirone; and ginkgo biloba (6). An example of combination therapy would include adding acetylcholinesterase inhibitors to sildafenil, a treatment for erectile dysfunction (77); sidenafil increases genital blood flow but may not impact on subjective reports of arousal (16). Furthermore, acetylcholinesterase inhibitors may augment sildafenil's actions on erectile desire in women (16) and another example would be acetylcholinesterase inhibitors added to oral or transdermal testosterone to augment its libido effects in women or men.

The phrase “therapeutically effective amount” relates to amounts of compounds or compositions that produce a desired therapeutic benefit in patients that is in excess of the side effect (adverse effect) rates noted in patients for compounds or compositions useful in this invention. “Therapeutically effective amount” or “pharmacologically effective” can also refer to that amount of a compound, or composition, effective to produce an “intended pharmacological result”, e.g., treat or reverse sexual dysfunction associated with medication-, substance-, disease-, and other medical conditions. For males and/or females, “intended pharmacological results” can include increases in: sexual drive; fantasizing about sex; interest in sexually explicit material; frequency of sexual activity; masturbation; rates of orgasm; rates of sexual arousal. For males, “intended pharmacological results” can also include erection vigor or spontaneous erections. For females, “intended pharmacological results” can further include increase vaginal lubrication. “Intended pharmacological results” can also include improvements in the assessment of an individual's sexual life (as indicated in Tables 1-3), including increase satisfaction with sexual functioning and enjoyment of the individual's sexual romantic life. For example, increases in the numerical scoring associated with items 1-4, 6, 7, 9, 12, and 13 (for men and women as assessed by Tables 2-3) are an “intended pharmacological result” in accordance with the subject invention. Likewise, decreases in the scoring associated with items 5, 8, 10, and 11 are an “intended pharmacological result” of the subject invention.

The response rate (or observation of one or more intended pharmacological result) for compounds used in the practice of the subject invention is, typically at least: 0.1%, 0.5%, or 1% of the patient population. For example, the response rate (or observation of one or more intended pharmacological result) for patients treated with donepezil is, typically, greater than 1%. Alternatively, the response rate (or observation of one or more intended pharmacological result) to donepezil is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent or higher.

For galantamine, the response rate (or observation of one or more intended pharmacological result) to treatment is greater than 0.1% to 1% of the patient population. In various embodiments, the response rate (or observation of one or more intended pharmacological result) is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent or higher.

Where rivastigmine is used in the practice of the instant invention, the response rate (or observation of one or more intended pharmacological result) to the therapeutic protocol is at least 0.1% to 1% of the patient population. Other embodiments provided for response rates (or observation of one or more intended pharmacological results) of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent or higher.

Response rates (or observation of one or more intended pharmacological result) associated with tacrine are at least 0.1% to 1% of the patient population. Alternatively, response rates/observation of one or more intended pharmacological results of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent or higher are provided by the subject invention.

Response rates (or observation of one or more intended pharmacological result) to neostigmine, physostigmine, and/or huperzine A are at least 0.1% to 1% of the patient population. Some embodiments provide for response rates (or observation of one or more intended pharmacological result) of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent or higher.

Compositions, useful in the methods of the invention, comprise one or more of the compounds (selected from the group consisting of donepezil, galantamine, tacrine, eptastigmine, physostigmine, rivastigmine, metrifonate, neostigmine, huperzine A, and combinations thereof) admixed with a pharmaceutical carrier. The compositions may be made according to conventional pharmaceutical compounding techniques. Thus, the carrier may take a wide variety of forms depending on the form of preparation desired for administration, e.g., injection, oral, suppository, topical, or potential. In various embodiments, compositions according to the subject invention comprise a pharmaceutical carrier and at least one compound. Yet other embodiments provide compositions containing a pharmaceutical carrier and two, three, four, five, six, seven, eight, or nine of the aforementioned compounds.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like.

Because of their ease in administration, tablets and capsules represent the most advantageous oral dosage unit form, in which case solid pharmaceutical carriers are employed. If desired, tablets may be sugar coated or enteric coated by standard techniques. Suppositories may be prepared, in which case cocoa butter could be used as the carrier.

In accordance with the invention, pharmaceutical compositions comprise, as an inactive ingredient, an effective amount of one or more non-toxic, pharmaceutically acceptable ingredient(s). Examples of such ingredients for use in the compositions include ethanol, dimethyl sulfoxide, glycerol, silica, alumina, starch, calcium carbonate, talc, flour, and equivalent non-toxic carriers and diluents.

Where the pharmaceutical compositions are aerosols, the active ingredients can be packaged in pressurized aerosol containers with a propellant (e.g., carbon dioxide, nitrogen, propane) admixed with the usual adjuvants (such as co-solvents or wetting agents).

The pharmaceutical compositions herein will contain, per dosage unit (e.g., tablet, capsule, powder, injectable, teaspoon, suppository, bandage, and the like) active ingredients (i.e., drugs useful for the treatment of a disorder) in amounts of about 0.1 to about 100 mg of the active ingredient. In a preferred embodiment, the compositions comprise about 0.1 and 50 mg per dosage unit. Other embodiments provide for a dose of active ingredient of about: 0.5 to 25 mg per dosage unit; 0.75 to 12.5 mg per dosage unit; 1 to 7.5 mg per dosage unit; or 1.25 to 5 mg per dosage unit. In the context of this paragraph, the term “about” can be substituted with “at least about” or “at least.”

Tablets can contain the following inactive ingredients: lactose hydrous, pregelatinized starch, microcrystalline cellulose, sodium starch glycolate, magnesium stearate, purified water, carnauba wax, hydroxypropyl methyl cellulose, titanium dioxide, polyethylene glycol, synthetic iron oxide, and polysorbate 80.

A “pharmaceutical composition” refers to a composition suitable for pharmaceutical use in a subject. The pharmaceutical compositions of this invention comprise a pharmacologically effective amount of a compound of the invention and a pharmaceutically acceptable carrier. The terms “pharmaceutically acceptable carrier” or “pharmaceutical carrier” refers to any of the standard pharmaceutical carriers, buffers, and excipients, such as a phosphate buffered saline solution, aqueous solutions of dextrose, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Preferred pharmaceutical carriers can be selected by those of skill in the art and generally depend upon the intended mode of administration of the active agent.

The compositions of the invention can be formulated for administration in a variety of ways. Typical routes of administration include, for example, oral, enteral and parenteral. These include, without limitation, subcutaneous, intramuscular, intravenous, intraperitoneal, intramedullary, intrapericardiac, intrabursal, oral, sublingual, ocular, nasal, topical, transdermal, transmucosal, or anal. The mode of administration can be, for example, via swallowing, inhalation, injection or topical application to a surface (e.g., eyes, mucus membrane, or skin).

Examples of aqueous solutions include, for example, water, saline, phosphate buffered saline, Hank's solution, Ringer's solution, dextrose/saline, glucose solutions and the like. The compositions can contain pharmaceutically acceptable auxiliary substances as required to approximate physiological conditions or to improve stability, appearance or ease of administration, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or sterile filtered. The resulting aqueous solutions can be packaged for use, as is, or lyophilized, the lyophilized preparation being combined with a sterile aqueous solution prior to administration.

Aqueous solutions are appropriate for injection and, in particular, for intravenous injection. The intravenous solution can include detergents and emulsifiers such as lipids. Aqueous solutions also are useful for oral and enteral and other routes of administration as tonics and administration to mucous or other membranes as, e.g., nose or eye drops. The carrier can be selected from various oils including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, maltose, rice, flour, chalk, silica gel, magnesium stearate, sodium stearate, glycerol monostearate, sodium chloride, dried skim milk, glycerol, propylene glycol, water, ethanol, and the like.

Systemic administration can also be by known transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be through nasal sprays, for example, or using suppositories.

For topical administration, the agents are formulated into ointments, creams, salves, powders and gels. In one embodiment, the transdermal delivery agent can be DMSO. Transdermal delivery systems can include, e.g., patches.

For inhalation, the compound is preferably administered in the form of an aerosol, liquid or solid. For aerosol administration, the compound preferably is supplied in finely divided form along with a surfactant and propellant. A surfactant may be required if the agent is immiscible in the propellant.

The surfactant preferably is soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride such as, for example, ethylene glycol, glycerol, erythritol, arabitol, mannitol, sorbitol, the hexitol anhydrides derived from sorbitol, and the polyoxyethylene and polyoxypropylene derivatives of these esters. Mixed esters, such as mixed or natural glycerides, can be employed. The surfactant can constitute 0.1%-20% by weight of the composition, preferably 0.25%-5%.

The balance of the aerosol composition is ordinarily propellant. Liquefied propellants are typically gases at ambient conditions, and are condensed under pressure. Among suitable liquefied propellants are the lower alkanes containing up to 5 carbons, such as butane and propane; and preferably fluorinated or fluorochlorinated alkanes. Mixtures of the above can also be employed. In producing the aerosol, a container equipped with a suitable valve is filled with the appropriate propellant, containing the agent as a solution or as finely divided particles and surfactant. The ingredients are thus maintained at an elevated pressure until released by action of the valve.

A nebulizer or aerosolizer device for administering compounds typically delivers a dose of active ingredient of about 0.1 and 50 mg per inhalation. Other embodiments provide for a dose of active ingredient of about: 0.5 to 25 mg per inhalation; 0.75 to 12.5 mg per inhalation; 1 to 7.5 mg per inhalation; or 1.25 to 5 mg per inhalation.

The subject invention does not include within its scope: a) the treatment of physiologic male erectile dysfunction by the administration of galanthamine; b) the treatment of sexual dysfunction in mammals comprising the administration of effective amounts of histamine receptor agonists (e.g., H₂ and/or H₃ agonists); or the treatment of psychogenic/psychological impotence comprising the administration of neostigmine bromide or neostigmine methyl sulfate. The invention also excludes the co-administration of histamine receptor (e.g., H₂ and/or H₃)

It should be understood that the examples and embodiments described herein are for illustrative purposes only and that various modifications or changes in light thereof will be suggested to persons skilled in the art and are to be included within the spirit and purview of this application and the scope of the appended claims. All publications and patents cited herein are hereby incorporated by reference in their entireties. All percentages are by weight and all solvent mixture proportions are by volume unless otherwise noted.

TABLE 1
SEXUAL FUNCTION QUESTIONNAIRE ITEMS
DRIVE/DESIRE FOR BOTH MEN AND WOMEN

| | | |
|---|---|---|
| STATUS OF SEXUAL DRIVE | INCREASE | DECREASE |
| FANTASIZING ABOUT SEX | INCREASE | DECREASE |
| INTEREST IN SEXUALLY EXPLICIT | INCREASE | DECREASE |
| MATERIAL | | |
| AMOUNT OF MASTURBATION | INCREASE | DECREASE |

AROUSAL/ORGASM FOR MEN

| | | |
|---|---|---|
| ERECTION VIGOR | INCREASE | DECREASE |
| FREQUENCY OF SPONTANEOUS | INCREASE | DECREASE |
| ERECTIONS | | |
| FREQUENCY OF PREMATURE | INCREASE | DECREASE |
| EJACULATION | | |
| FREQUENCY OF DELAYED | INCREASE | DECREASE |
| EJACULATION | | |

AROUSAL/ORGASM FOR WOMEN

| | | |
|---|---|---|
| FREQUENCY OF SEXUAL AROUSAL | INCREASE | DECREASE |
| ABILITY TO ACHIEVE VAGINAL | INCREASE | DECREASE |
| LUBRICATION | | |
| ABILITY TO ACHIEVE ORGASM | INCREASE | DECREASE |

SEXUAL ACTIVITY ASSESSMENT FOR BOTH MEN AND WOMEN

| | | |
|---|---|---|
| FREQUENCY OF INVOLVEMENT IN | INCREASE | DECREASE |
| SEXUAL ACTIVITY, WITH OR | | |
| WITHOUT A PARTNER, IN THE LAST | | |
| MONTH | | |

TABLE 2
SEXUAL FUNCTION QUESTIONNAIRE ITEMS
(Men)

Both sexual interest and activity can be affected by medications, legal and illegal substances, diseases, and other
medical conditions. Some of these medications, legal and illegal substances, diseases, and other medical conditions
can also alter sexual interest and activity.
I am going to ask you a few questions about different aspects of your sexual life.
DURING THE PAST TWO WEEKS HOW OFTEN DID YOU:

| | | NEVER | RARELY | SOMETIMES | OFTEN | VERY OFTEN |
|---|---|---|---|---|---|---|
| (0) | | (1-2) | (3-4) | (5-9) | (≧10) | |
| DESIRE | | | | | | |
| 1. | Experience a desire for sexual activity | (0) | (1) | (2) | (3) | (4) |
| 2. | Fantasize about sexual activity | (0) | (1) | (2) | (3) | (4) |
| 3. | Have an interest in initiating sexual activity | (0) | (1) | (2) | (3) | (4) |
| 4. | Have the ability to feel sexually excited (“turned on”) | (0) | (1) | (2) | (3) | (4) |

AROUSAL

| | | | | | | |
|---|---|---|---|---|---|---|
| 5. | Have difficulty getting an erection | (0) | (1) | (2) | (3) | (4) |
| 6. | Have an erection without being touched | (0) | (1) | (2) | (3) | (4) |
| 7. | Engage in sexual activity (intercourse or masturbation) | (0) | (1) | (2) | (3) | (4) |
| 8. | Have difficulty staying erect during sexual activity | (0) | (1) | (2) | (3) | (4) |

ORGASM

| | | | | | | |
|---|---|---|---|---|---|---|
| 9. | Experience an orgasm | (0) | (1) | (2) | (3) | (4) |
| 10. | Take longer than you would like to ejaculate | (0) | (1) | (2) | (3) | (4) |
| 11. | Fail to ejaculate during sexual activity | (0) | (1) | (2) | (3) | (4) |

SATISFACTION
OVERALL, if you have to give a number between 0 (worst case) and 10 (best case)
how would you describe:

| | | |
|---|---|---|
| 12. | Satisfaction with your sexual functioning | □ |
| 13. | Enjoyment of your sexual romantic life | □ |

TABLE 3
SEXUAL FUNCTION QUESTIONNAIRE ITEMS
(Women)

Both sexual interest and activity can be affected by medications, legal and illegal substances, diseases, and other
medical conditions. Some of these medications, legal and illegal substances, diseases, and other medical conditions
can also alter sexual interest and activity.
I am going to ask you a few questions about different aspects of your sexual life.
DURING THE PAST TWO WEEKS HOW OFTEN DID YOU:

| | | NEVER | RARELY | SOMETIMES | OFTEN | VERY OFTEN |
|---|---|---|---|---|---|---|
| (0) | | (1-2) | (3-4) | (5-9) | (≧10) | |
| DESIRE | | | | | | |
| 1. | Experience a desire for sexual activity | (0) | (1) | (2) | (3) | (4) |
| 2. | Fantasize about sexual activity | (0) | (1) | (2) | (3) | (4) |
| 3. | Have an interest in initiating sexual activity | (0) | (1) | (2) | (3) | (4) |
| 4. | Have the ability to feel sexually excited (“turned on”) | (0) | (1) | (2) | (3) | (4) |

AROUSAL

| | | | | | | |
|---|---|---|---|---|---|---|
| 5. | Have difficulty with vaginal lubrication | (0) | (1) | (2) | (3) | (4) |
| 6. | Become aroused without being touched | (0) | (1) | (2) | (3) | (4) |
| 7. | Engage in sexual activity (intercourse or masturbation) | (0) | (1) | (2) | (3) | (4) |
| 8. | Have difficulty staying lubricated during sexual activity | (0) | (1) | (2) | (3) | (4) |

ORGASM

| | | | | | | |
|---|---|---|---|---|---|---|
| 9. | Experience an orgasm | (0) | (1) | (2) | (3) | (4) |
| 10. | Take longer than you would like to have an orgasm | (0) | (1) | (2) | (3) | (4) |
| 11. | Fail to have an orgasm during sexual activity | (0) | (1) | (2) | (3) | (4) |

SATISFACTION
OVERALL, if you have to give a number between 0 (worst case) and 10 (best case)
how would you describe:

| | | |
|---|---|---|
| 12. | Satisfaction with your sexual functioning | □ |
| 13. | Enjoyment of your sexual romantic life | □ |

### REFERENCES

1. Finger, W. W.; Lund, M.; Slagle, M. A. [1997] “Medications that may contribute to sexual disorders: A guide to assessment and treatment in family practice,” J Fam Pract 44:33-43.

2. Sadock, V. A. [1995] “Normal Human Sexuality and Sexual and Gender Identity Disorders: Normal Human Sexuality and Sexual Dysfunction,” Comprehensive Textbook of Psychiatry, Baltimore, Md.: Kaplan H I, Sadock B J (eds)., p. 1305, Williams and Wilkins.

3. Meston, C. M.; Frohlich, P. F. [2000] “The neurobiology of sexual function,” Arch Gen Psychiatry 57:1012-1030.

4. Woosley, R. L.; Flockhart, D. [1995] “A case for the development of Centers for Education and Research in Therapeutics (C.E.R.T.),” Pharmacy Week 4:1-3.

5. Watts, R. J. [1982] “Sexual functioning, health beliefs, and compliance with high blood pressure medications,” Nurs Res 31:278-283.

6. Rosen, R. C.; Lane, R. M.; Menza, M. [1999] “Effects of SSRIs on sexual function: A critical review,” J Clin Psychopharm 19:67-85.

7. Tanner, L. A.; Hoff, R.; Carmichael, L. P. [1976] “Teaching sex education and counseling for the primary physician,” South Med J 69:1591-1594.

8. Blanco, R.; Saenz de Tejada, I.; Goldstein, I.; Krane, R. J.; Wotiz, H. H.; Cohen, R. A. [1988] “Cholinergic neurotransmission in human corpus cavernosum, II: acetylcholine synthesis,” Am J Physiol 254:H468-H472.

9. Smirai, M.; Sadaki, K.; Rikimaru, A. [1972] “Biochemical investigation on the distribution of adrenergic and cholinergic nerves in human penis,” Tohoku J Exp Med 104:403-404.

10. Adaikan, P. G.; Karim, S. M. M.; Kottegoda, S.R.; Ratnam, S. S. [1983] “Cholinoreceptors in the corpus cavernosum of the human penis,” J Auton Pharmacol 3:107-111.

11. Godec, C. J.; Bates, H. [1984] “Cholinergic receptors in corpus cavernosa,” Urology 24:31-33.

12. Saenz de Tejada, I.; Blanco, R.; Goldstein, I.; Azadzoi, K.; de las Morenas, A.; Krane, R. J.; Cohen, R. A. [1988] “Cholinergic neurotransmission in human corpus cavernosum, I: responses of isolated tissue,” Am J Physiol 254:H459-467.

13. Klinge, E.; Sjostrand, N. O. [1977] “Comparative study of some isolated mammalian smooth muscle effectors of penile erection,” Acta Physiol Scan 100:354-367.

14. Hyyppa, M. T.; Rinne, U. K.; Sonninen, V. [1970] “The activating effect of L-dopa treatment on sexual functions and its experimental background,” Acta Neurolog Scand 2:177-179.

15. Levine, L. A. [2000] “Erectile dysfunction: a review of a common problem in rapid evolution,” Prim Care Update Ob Gyns 7:124-129.

16. Meston, C. M.; Frohlich, P. F. [2001] “Update on female sexual function,” Curr Opin Urol 11:603-609.

17. Guay, A. T. [2001] “Lecture 5: Sexual dysfunction in the diabetic patient,” Int J Impot Res 13 (Suppl 5):S47-50.

18. Saenz de Tejada, I.; Goldstein, I.; Azadzoi, K.; Krane, R. J.; Cohen, R. A. [1989] “Impaired neurogenic and endothelium-mediated relaxation of penile smooth muscle from diabetic men with impotence,” N Engl J Med 320:1025-1030.

19. Knispel, H. H.; Goessl, C.; Beckmann, R. [1992] “Nitric oxide mediates relaxation in rabbit and human corpus cavernosum smooth muscle,” Urol Res 20:253-257.

20. Kim, J. H.; Klyachkin, M. L.; Svendsen, E.; Davies, M. G.; Hagen, P. O.; Carson, C. C. [1994 ] “3rd: Experimental hypercholesterolemia in rabbits induces cavernosal atherosclerosis with endothelial and smooth muscle cell dysfunction,” J Urol 151:198-205.

21. Srilatha, B.; Adaikan, P. G.; Ng, S. C.; Arulkumaran, S. [1999] “Elevated low-density lipoprotein cholesterol (LDL-C) enhances pro-erectile neurotransmission in the corpus cavernosum,” Int J Impot Res 11: 159-165.

22. Segraves, R. T. [1989] “Effects of psychotropic drugs on human erection and ejaculation,” Arch Gen Psychiatry 46:275-284.

23. Wagner, G.; Brindley, G. S. [1980] “The effect of atropine and B blockers on human penile erection: A controlled pilot study,” Vasculogenic Impotence, Zorgniott A, Rossi G (eds), pp 77-82, Charles C Thomas Publishers, Springfield, Ill.

24. Wagner, G.; Levin, R. J. [1980] “Effect of atropine and methylatropine on human vaginal blood flow, sexual arousal and climax,” Acta Pharmacol Toxicol 46:321-325.

25. Riley, A. J.; Riley, E. J. [1983] “Cholinergic and adrenergic control of human sexual responses,” Psychopharmacology and Sexual Disorders, Wheatley D (ed), pp 125-137, Oxford University Press Inc., New York.

26. Penttila, O. [1966] “Acetylcholine, biogenic amines and enzymes involved in their metabolism in penile erectile tissue,” Ann Med Exp Biol Fenn 44(suppl 9):7-42.

27. Segraves, R. T. [1987] “Bethanechol reversal of imipramine-induced ejaculatory dysfunction (letter)” Am J Psychiatry 144:1243.

28. Yager, J. [1986] “Bethanecol chloride can reverse erectile and ejaculatory dysfunction induced by tricyclic antidepressants and mazindol: case report,” J Clin Psychiatry 47:210-211.

29. Gross, M. D. [1982] “Reversal by bethanechol of sexual dysfunction caused by anticholinergic antidepressants,” Am J Psychiatry 139:1193-1194.

30. Hull, E. M.; Bitran, D.; Pehek, E. A.; Holmes, G. M.; Warner, R. K.; Band, L. C.; Clemens, L. G. [1988] “Brain localization of cholinergic influence on male sex behavior in rats: agonists,” Pharmacol Biochem Behav 31:169-174.

31. Hull, E. M.; Pehek, E. A.; Bitran, D.; Holmes, G. M.; Warner, R. K.; Band, L. C.; Bazzett, T.; Clemens, L. G. [1988] “Brain localization of cholinergic influence on male sex behavior in rats: antagonists,” Pharmacol Biochem Behav 31:175-178.

32. Seagraves, R. T. [1987] “Reversal by bethanechol of imipramine-induced ejaculatory dysfunction,” Am J Psychiatry 144:1243.

33. Sorscher, S. M.; Dilsaver, S. C. [1986] “Antidepressant-induced sexual dysfunction in men: Due to cholinergic blockade?” J Clin Psychopharmacol 6:53-55.

34. Kleeman, F. J. [1970] “The physiology of the internal sphincter,” J Urol 104:549-554.

35. Burnett, A. L.; Lowenstein, C. J.; Bredt, D. S.; Chang, T. S. K.; Snyder, S. H. [1992] “Nitric oxide: a physiological mediator of penile erection,” Science 257:401-403.

36. Aydin, S.; Ozbek, H.; Yilmaz, Y.; Atilla, M. K.; Bayrakli, H.; Cetin, H. [2001] “Effects of sildenafil citrate, acetylcholine, and sodium nitroprusside on the relaxation of rabbit cavernosal tissue in vitro,” Urology 58:119-124.

37. Heath, R. G. [1972] “Pleasure and brain activity in man,” J Nerv Mental Dis 154:3-10.

38. Hersi, A. I.; Kitaichi, K.; Srivastava, L. K.; Gaudreau, P.; Quirion, R. [2000] “Dopamine D-5 receptor modulates hippocampal acetylcholine release,” Mol Brain Res 76:336-340.

39. Isa, Y. [1995] “Repeated MK-801 administration but not methamphetamine produces up-regulation of muscarinic acetylcholine receptors in mice,” Masui 44:1648-1653.

40. Courty, E.; Durif, F.; Zenut, M.; Copurty, P.; Laverenne, J. [1997] “Psychiatric and sexual disorders induced by apomorphine in Parkinson's disease,” Clin Neuropharmacol 20:140-147.

41. Uitti, R. J.; Tanner, C. M.; Rajput, A. H.; Goetz, C. G.; Klawans, H. L.; Thiessen, B. [1989] “Hypersexuality with antiparkinsonian therapy,” Clin Neuropharmacol 12:375-383.

42. Goodwin, F. K. [1971] “Psychiatric side effects of levodopa in man,” JAMA 218:1915-20.

43. Melis, M. R.; Argiolas, A. [1995] “Dopamine and sexual behavior,” Neurosci Biobehav Rev 19:19-38.

44. Bowers, M. B.; Woert, M. V.; Davis, L. [1971] “Sexual behavior during L-dopa treatment for Parkinsonism,” Am J Psychiatry 127:1691-1693.

45. Hyyppa, M.; Rinne, U. K.; Sonninen, V. [1970] “The activating effect of L-dopa treatment on sexual functions and its experimental background,” Acta Neurol Scand 46(suppl 43):223 & 43(suppl 46):223-224.

46. O'Brien, C. P.; DiGiacomo, J. N.; Fahn, S.; Schwarz, G. A. [1971] “Mental effects of high-dosage levodopa,” Arch Gen Psychiatry 24:61-64.

47. Danjou, P.; Alexandre, L.; Warot, D.; Lacomblez, L.; Puech, A. J. [1988] “Assessment of erectogenic properties of apomorphine and yohimbine in man,” Br J Clin Pharmacol 26:733-739.

48. Danjou, P.; Lacombiez, L.; Warot, D.; Peuch, A. J. [1989] “Assessment of erectogenic drugs by numeric plethysmography,” J Pharmacol Methods 21:61-69.

49. Lal, S.; Ackman, D.; Thavundayil, J. X.; Kiely, M. E.; Etienne, P. [1984] “Effect of apomorphine, a dopamine receptor agonist, on penile tumescence in normal subjects,” Prog Neuropsychopharmacol Biol Psychiatry 8:695-699.

50. O'Sullivan, J. D.; Hughes, A. J. [1998] “Apomorphine-induced penile erections in Parkinson's disease,” Mov Disord. 13:536-539.

51. Segraves, R. T.; Bari, M.; Segraves, K.; Spirnak, P. [1991] “Effect of apomorphine on penile tumescence in men with psychogenic impotence,” J Urol 145:1174-1175.

52. Lal, S.; Laryea, E.; Thavundayil, J. X.; Nair, N. P. V.; Negrete, J.; Ackman, D.; Blundell, P.; Gardiner, R. J. [1987] “Apomorphine-induced penile tumescence in impotent patients preliminary findings,” Prog Neuropsychopharmacol Biol Psychiatry 11:235-242.

53. Lal, S.; Tesfaye, Y.; Thavundayil, J. X.; Thompson, T. R.; Kiely, M. E.; Nair, N. P. V.; Grassino, A.; Dubrovsky, B. [1989] “Apomorphine: clinical studies on erectile impotence and yawning,” Prog Neuropsychopharmacol Biol Psychiatry 13:329-339.

54. Clark, J. T.; Stefanick, M. L.; Smnith, E. R.; Davidson, J. M. [1983] “Further studies on alterations in male rat copulatory behavior induced by the dopamine-receptor agonists RDS-127,” Pharmacol Biochem Behav 19:781-786.

55. Foreman, M. M.; Hall, J. L. [1987] “Effects of D₂-dopaminergic receptor stimulation on male rat sexual behavior,” J Neural Transm 68:153-170.

56. Hull, E. M.; Bitran, D.; Pehek, E. A.; Warner, R. K.; Band, L. C.; Holmes, G. M. [1986] “Dopaminergic control of male sex behavior in rats: effects of an intracerebrally-infused agonist,” Brain Res 370:73-81.

57. Mas, M.; Fumero, B.; Perez-Rodriquez, I. [1995] “Induction of mating behavior by apomorphine in sexually sated rats,” Eur J Pharmacol 280:331-334.

58. Arnold, H. M.; Fadel, J.; Sarter, M.; Bruno, J. P. [2001] “Amphetamine-stimulated cortical acetylcholine release: role of the basal forebrain,” Brain Res 894:74-87.

59. Ghadirian, A.M.; Chouinard, G.; Annable, L. [1982] “Sexual dysfunction and plasma prolactin levels in neuroleptic-treated schizophrenic outpatients,” J Nerv. Ment Dis 170:463-467.

60. Shen, W. W.; Sata, L. S. [1983] “Inhibited female orgasm resulting from psychotropic drugs: a clinical review,” J Reprod Med 28:497-499.

61. Shen, W. W.; Sata, L. S. [1990] “Inhibited female orgasm resulting from psychotropic drugs: a five-year, updated, clinical review,” J Reprod Med 35:11-14.

62. Richelson, E. [1994] “The pharmacology of antidepressants at the synapse: focus on newer compounds,” J Clin Psychiatry 55(suppl A):34-39.

63. Kiefer, G.; Fischer, W.; Feuerstein, T. J. [1999] “Effects of amitriptyline, amitriptylinoxide, doxepine and clozapine on N-methyl-D-aspartate-evoked release of [3H]-acetylcholine in rat caudatoputamen,” Arzneimittelforschung 49:820-823.

64. Gwee, M. C.; Cheah, L. S. [1986] “Actions of cimetidine and ranitidine at some cholinergic sites: implications in toxicology and anesthesia,” Life Sci 39:383-388.

65. Hennings, E. C. P.; Kiss, J. P.; Vizi, E. S. [1997] “Nicotinic acetylcholine receptor antagonist effectg of Fluoxetine in rat hippocampal slices,” Brain Research 292-294.

66. Fryer, J. D.; Lukas, R. J. [1999] “Antidepressants noncompetitively inhibit nicotinic acetylcholine receptor function,” J Neurochem 72:1117-1124.

67. Dazzi, L.; Vacca, G.; Ladu, S.; Pisu, M. G.; Serra, M.; Biggio, G. [2001] “Long-term treatment with antidepressant drugs reduces the sensitivity of cortical cholinergic neurons to the activating actions of stress and the anxiogenic drug FG 7142,” Neuropharmacology 41:229-237.

68. “Effect of venlafaxine hydrochloride in different preparations of isolated guinea-pig and rat organ tissues,” J Auton Pharmacol [1999] 19:109-113.

69. Slemmer, J. E.; Martin, B. R.; Damaj, M. I. [2000] “Bupropion is a nicotinic antagonist,” J Pharmacol Exp Ther 295:321-327.

70. Sherman, K. A.; Zigmond, M. J.; Hanin, I. [1981] “Fluphenazine-induced decline in striatal acetylcholine content in not abolished by exogenous choline,” Neuropharmacology 20:921-924.

71. Arendt, T. [1994] “Impairment in memory function and neurodegenerative changes in the cholinergic basal forebrain system induced by chronic intake of ethanol,” J Neural Transm Suppl 44:173-187.

72. Gazyakan, E.; Hennegriff, M.; Haaf, A.; Landwehrmeyer, G. B.; Feuerstein, T. J.; Jackisch, R. [2000] “Characterization of opioid receptor types modulating acetylcholine release in septal regions of the rat brain,” Naunyn Schmiedebergs Arch Pharmacol 362:32-40.

73. Yates, S. L.; Bencherif, M.; Fluhler, E. N.; Lippiello, P. M. [1995] “Up-regulation of nicotinic acetylcholine receptors following chronic exposure of rats to mainstream cigarette smoke or alpha 4 beta 2 receptors to nicotine,” Biochem Pharmacol 22;50:2001-2008.

74. Kamiya, Y.; Andoh, T.; Watanabe, I.; Higashi, T.; Itoh, H. [2001] “Inhibitory effects of barbiturates on nicotinic acetylcholine receptors in rat central nervous system neurons,” Anesthesiology 94:694-704.

75. Shih, T. M.; Hanin, I. [1978] “Effects of chronic lead exposure on levels of acetylcholine and choline and on acetylcholine turnover rate in rat brain areas in vivo,” Psychopharmacology (Berl) 58:263-269.

76. Imbimbo, B. P. [2001] “Pharmacodynamic-tolerability relationships of cholinesterase inhibitors for Alzheimer's disease,” CNS Drugs 15:375-390.

77. Physicians Desk Reference, 2002.

78. Bai, D. L.; Tang, X. C.; He, X. C. [2000] “Huperzine A, a potent therapeutic agent for treament of Alzheimer's disease,” Curr Med Chem 7:355-374.

79. Hanin, I.; Tang, X. C.; Kindel, G. L.; Kozikowski, A. P. [1993] “Natural and synthetic Huperzine A: effect on cholinergic function in vitro and in vivo,” Ann NY Acad Sci 695:304-306.

80. Scott, L. J.; Goa, K. L. [2000] “Galantamine: a review of its use in Alzheimer's disease,” Drugs 60:1095-1122.

81. Coyle, J.; Kershaw, P. [2001] “Galantamine, a cholinesterase inhibitor that allosterically modulates nicotinic receptors: effects on the course of Alzheimer's disease,” Biol Psychiatry 49:289-299.

82. Yu, Q.; Holloway, H. W.; Flippen-Anderson, J. L.; Hoffman, B.; Brossi, A.; Greig, N. H. [2001] “Methyl analogues of the experimental Alzheimer drug phenserine: synthesis and structure/activity relationships for acetyl- and butyrlcholinesterase inhibitory action,” J Med Chem 44:4062-4071.

83. Doucet-Personeni, C.; Bentley, P. D.; Fletcher, R. J.; Kinkaid, A.; Kryger, G.; Pirard, B.; Taylor, A.; Taylor, R.; Taylor, J.; Viner, R.; Silman, I.; Sussman, J. L.; Greenblatt, H. M.; Lewis, T. [2001] “A structure-based design approach to the development of novel, reversible AchE inhibitors,” J Med Chem 44:3203-3215.

84. Dow, J.; Dulery, B. D.; Harnsperger, J. M.; DiFrancesco, G. F.; Keshary, P.; Haegele, K. D. [1995 ] “Pharmacokinetics and pharmacodynamics of the acetylcholinesterase inhibitor 2,2,2-trifluoro-1-(3-trimethylsilylphenyl) ethanone in dog. Potential for transdermal patch delivery,” Arzneimittelforschung 45:1245-1252.

85. Moriearty, P. L.; Thornton, S. L.; Becker, R. E. [1993] “Transdermal patch delivery of acetylcholiesterase inhibitors,” Methods Find Exp Clin Pharmacol 15:407-412.

86. Benech, H.; Vincenti, M.; Fouchart, F.; Pruvost, A.; Vienet, R.; Istin, M.; Grognet, J. M. [1998] “Development and in vivo assessment of a Transdermal system for physostigmine,” Methods Find Exp Clin Pharmacol 20:489-498.

87. Dohanich, G. P.; Cada, D. A. [1989] “Reversal of androgen inhibition of estrogen-activated sexual behavior by cholinergic agents,” Horm Behav 23:503-513.

88. Blockmans, D.; Steeno, O. [1988] “Physostigmine as a treatment for anejaculation with paraplegic men,” Andrologia 20:311-313.

89. Otani, T.; Kondo, A.; Takita, T. [1986] “A paraplegic fathering a child after an intrathecal injection of neostigmine: case report,” Paraplegia 24:32-37.

90. Linsenmeyer, T. A.; Perkash, I. [1991] “Infertility in men with spinal cord injury,” Arch Phys Med Rehabil 72:747-754.

91. Kennedy, S. H.; Dickens, S.; Eisfeld, B. S., et al. [1999] “Sexual dysfunction before anti-depressant therapy in major depression,” J Affect Disord 56:197-204.

1. A method for the treatment of medication-, substance-, disease-, and/or other medical condition-related sexual dysfunction comprising the administration of a therapeutically effective amount of at least one acetylcholinesterase inhibitor to an individual, wherein:
a) said at least one acetylcholinesterase inhibitor is not co-administered with histamine receptor agonists; and
b) said method excludes: i) the treatment of physiologic male erectile dysfunction comprising the administration of galanthamine; and ii) the treatment of psychogenic/psychological impotence comprising the administration of neostigmine bromide or neostigmine methyl sulfate.

2. The method according to claim 1, wherein said sexual dysfunction is selected from the group consisting of reduced sexual desire, delayed or inhibited ejaculation, delayed orgasm, anorgasmia, and erectile disorders.

3. The method according to claim 1, wherein said acetylcholinesterase inhibitor is selected from the group consisting of donepezil, galantamine, tacrine, eptastigmine, physostigmine, rivastigmine, metrifonate, neostigmine, huperzine A, and combinations thereof.

4. The method according to claim 3, wherein two acetylcholinesterase inhibitors are selected.

5. The method according to claim 3, wherein three acetylcholinesterase inhibitors are selected.

6. The method according to claim 3, wherein four acetylcholinesterase inhibitors are selected.

7. The method according to claim 3, wherein five acetylcholinesterase inhibitors are selected.

8. The method according to claim 3, wherein six acetylcholinesterase inhibitors are selected.

9. The method according to claim 3, wherein seven acetylcholinesterase inhibitors are selected.

10. The method according to claim 3, wherein eight acetylcholinesterase inhibitors are selected.

11. The method according to claim 3, wherein nine acetylcholinesterase inhibitors are selected.

12. The method according to claim 1, wherein said acetylcholinesterase inhibitor is administered orally, transdermally, nasally, or mucosally.

13. The method according to claims 1, 2, 3, 4, 5, 6, 7, 8, 9, 10, 11, or 12, wherein said acetylcholinesterase inhibitor(s) is co-administered with an agent selected from the group consisting of sildenafil, tadalafil, vardenafil, cyproheptadine, tradodone, nefazodone, mianserin, mirtazapine, granisetron, yohimbine, amantadine, dextroamphetamine, pemoline, methylphenidate, bupropion, buspirone, ginkgo biloba, and various combinations of said agents.
